(19) United States
(12) Patent Application Publication
(10) Pub. No.: US 2012/0184756 A1
(43) Pub. Date: Jul. 19, 2012

(54) Production process of furfural from cellulose using as a solvent and catalyst an imidazolium sulphate or chloride in a reaction with simultaneous extraction with dibutyl ether

(76) Inventor: Pedro Brito Correia, (US)

(21) Appl. No.: 13/007,609

(22) Filed: Jan. 15, 2011

(51) Int. Cl. C07D 307/50

(52) U.S. Cl. 549/489

(57) ABSTRACT

Furfural is a known component of both gasoline or diesel fuels as well as a chemical raw material.

We disclose a new production process from cellulose, using as a solvent and catalyst an alkyl imidazole sulphate or chloride or a mixture of both in a reaction with simultaneous extraction, using as a solvent dibuthyl ether.

Furfural and dibuthyl ether are later separated by distillation.

### 1. FIELD OF INVENTION

Furfural, liquid biofuels from cellulose, ionic liquids, renewable energy, solvent extraction from ionic liquids

### 2. BACKGROUND OF THE INVENTION

Since many years the acid hydrolysis of cellulose was used in the industry to produce glucose. Glucose was converted to ethanol by enzymatic means. However, most of the ethanol from vegetables is produced from sugar from sugar cane and not from cellulose.

In fact, the acid hydrolysis of cellulose was only used in special cases of shortage of alternative fuels or as a pilot process.

The limitations where:
Finding a suitable solvent for cellulose to allow the acid hydrolysis. If the solvent is not adequate, the hydrolysis reaction takes place at the interphase of the cellulose solid particles and the solvent. Under these conditions, a higher crystallinity of cellulose as well as intermolecular hydrogen bonds reduce the reaction rate.
Avoiding the further dehydration of glucose to side products under the reaction conditions of the hydrolysis of cellulose. In fact, the dehydration of glucose and its degradation takes place at a 20-30° C. lower temperature than the cellulose hydrolysis. Under the cellulose hydrolysis conditions, glucose degrades very strongly.
Efforts have been made in the last 10 years:
   to hydrolyse cellulose in ionic liquids and several other polar organic solvents in order to approach the solution and the hydrolysis temperature of cellulose to the dehydration temperature of glucose
   to dehydrate hexoses like fructose and glucose to hydroxymethyl furalfural. In these processes, glucose is isomerised to fructose previously.
   to improve the dehydration of pentoses to furfural, which is an old process used intensively in the furfural production mainly for chemicals
Although much research has been done to improve each step of the conversion of cellulose to a liquid fuel, the two main difficulties were to dissolve and to hydrolyse cellulose with high yields under conditions where glucose is not degraded.
In our previous experiments, we used as an ionic liquid N-alkyl imidazole hydrochloride as it is the best known solvent for cellulose. We tried also with additional small quantities of hydrochloric, sulphuric and phosphoric acids. However, at the temperature of hydrolysis of cellulose, the velocity of dehydration of glucose and its degradation is too large to allow taking out from the reaction medium the dehydration products of glucose with acceptable yields.

### 2. DETAILED DESCRIPTION OF THE INVENTION

After trying many alternative ionic liquids, we succeeded to make the conversion of cellulose using methyl imidazole sulphate with small quantities of N-methyl imidazole hydrochloride, according to the following reactions.

We found that under our reaction conditions hydroxymethyl furfural is converted to furfural. To avoid the following dimerization of furfural to furoin, we added a small excess of sulphuric acid.

It is known that hydroxymethyl furfural is an unstable product, which must be stored at 5° C., and therefore reacts further namely to furfural or oligomers.

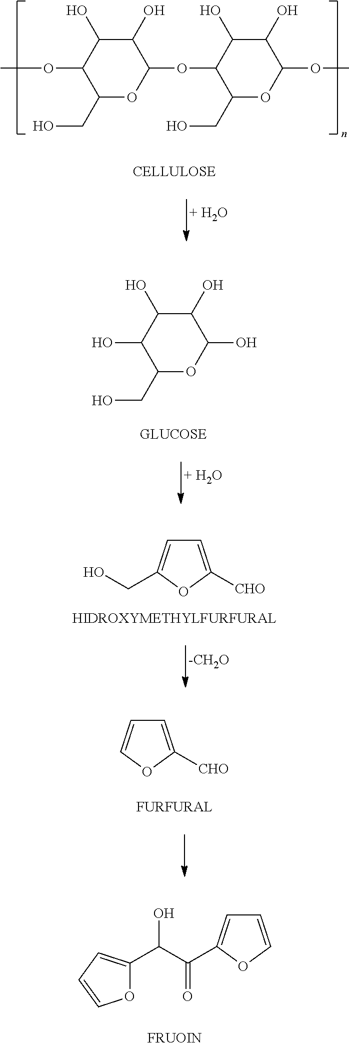

In the reaction medium N-alkyl imidazolium sulphate with a small quantity of N-alkyl imidazolium hydrochloride we used directly glucose instead of cellulose to study the kinetics of glucose dehydration and further degradation. We found that only 20% of glucose was converted into furfural, the rest giving side products due to further reaction of furfural under these reaction conditions. These side products could not be extracted by any ether and had a dark brown colour.

We concluded that the limitation to the yield of furfural from cellulose is not only the hydrolysis of cellulose but also the conversion of glucose to several side products, namely coloured oligomers.

We tried to remove furfural from the reaction medium by azeotrope distillation with water, a known process used in industry to produce furfural from pentoses dissolved in water or from carbohydrates containing pentoses.

However, we found that to remove furfural by azeotropic distillation it would be necessary to have a quantity of water in the reaction medium, which would reduce strongly the solubility of cellulose.

We also tried to make a stop and go reaction process, by hydrolysing cellulose, cooling and extracting furfural with a solvent. However, this process is more expensive and has lower yields than the continuous solvent extraction.

Therefore we tried a solvent with a boiling point near the reaction temperature at atmospheric pressure, which is dibuthyl ether.

We also tried to use as a solvent buthanol, which is described as a good solvent in the dehydration of fructose in organic solvents, but not described for extraction in ionic liquids.

In fact, buthanol cannot be used with ionic liquids containing alkyl imidazole, because buthanol extracts together with furfural a relevant quantity of N-alkyl imidazole.

After the reaction with extraction, we separated by distillation furfural (Boiling point 165° C.) from dibuthylether (boiling point 142° C.).

Furfural was identified and quantified by GC-MS and NMR.

The content of water during the hydrolysis of cellulose is critical to obtain good yields. A lower water content favours dehydration of glucose. A higher water content reduces the solubility of cellulose.

A higher temperature is favourable for the hydrolysis, but more side products are produced by dehydration. The concentration of cellulose in the reaction medium is also critical. The limit is the concentration of glucose, which may dehydrate intramolecular in a first order kinetics or intermolecular in a second order kinetics.

A higher concentration of glucose favours the intermolecular dehydration, which we want to avoid.

For the performance of the process it is important to consider that under the reaction conditions dibuthyl ether does not hydrolyse, and that the partition coefficient of the ionic phase to dibuthyl ether for furfural is 1 to 10.

### Example

In a round bottom flask we added:

| | |
|---|---|
| N-methyl imidazole | 0.5 mole-41.5 g |
| Hydrochloric acid 37% | 0.2 mole-20 g |
| Sulphuric acid | 0.4 mole-39.2 g |
| Celulose | 10.0 g |
| Dibuthyl ether | 50 g |

We heated with a reflux condenser with an oil bath at 160° C. under strong stirring during one hour. We separated the two phases.

The dibuthyl ether phase was distilled in a vigreux column to create reflux and several distillation plates.

We obtained 5.3 g of distillation residue, which contained 98% furfural, as identified by GC-MS and NMR.

### BIBLIOGRAPHY

1. Jaroslaw Lewkowski, Synthesis, Chemistry and Applications of 5-Hydroxymethyl-furfural and its derivatives, Arkivoc, 2001, 17-54
2. Claude Moreau, Annie Finiels, Laurent Vanoye, Dehydration of fructose and sucrose into 5-hydroxymethylfurfural in the presence of 1-H-3-methyl imidazolium chloride acting both as solvent and catalyst, journal of Molecular Catalysis A, 2006, 165-169
3. Fred Shafizedh, Saccharification of lignocellulosic materials, Pure and Appl. Chem., vol 55, No 4, pp 705-720, 1983
4. Khavinet Lourvanij and Gregory Rorrer, Reaction rates for the partial dehydration of glucose to organic acids in solid-acid molecular sieving catalyst powders J. Chem. Tech. Biotechnol., 1997, 69, 35-44
5. Yuri Roman Leshkov, Christopher Barrett, Zehn Y. Liu, James A. Dumesic, Production of dimethylfuran for liquid fuels from biomass derived carbohydrates, Nature, Vol 447, 21 Jun. 2007, 982
6. Acid in ionic liquid: an efficient system for hydrolysis of lignincellulose, Changzhi Li et al. Green Chemistry, 17 Dec. 2007
7. Catalytic conversion of cellulose into Sugar alcohols Atsushi Fukuoka et al. Angewandte Chemie, 2006, 45, 5161-5163
8. Pyranone by pyrolysis of cellulose, Fred Shafizadeh, Pure & Appliedd Chem, 1983, 55-4, 705-720
9. Dissolution of cellulose with ionic liquids and its application—a minireview, Shengdong Zhu et al, Green Chemistry, 2006, 8, 325-327
10. WO 2008/053284 A1—Liquid biofuels containing dihydroxymethyl furan, Pedro Correia, priority date 9 Mar. 2007.
11. PCT IB 2008 03313, Liquid biofuels containing 2 methyl tetrahydro pyran, Pedro Correia
12. USP application 123566643—Liquid biofuels from cellulose, Pedro Correia
13. USP application 12748425—Liquid biofuels from cellulose using trioctylamine hydrochloride, Pedro Correia
14. Simple chemical transformation of lignocellulosic biomass into furan for fuel and chemicals, J. Am. Chem. Soc. 2009, 131, (5), 1979-1985, Joseph Binder, Ronald Raines

1. A production process of furfural from wood, sugar cane directly, bagasse from sugar cane, cellulose or starch based on a reaction with simultaneous extraction, where the reaction medium contains as a first solvent and catalyst a mixture of N-alkylimidazolium sulphate, N-alkylimidazolium chloride, water, an additional amount of sulphuric acid, cellulose and as a second solvent dibuthylether, heated at a temperature of 80-200° C., during 30 minutes to 4 hours, separation of the phase containing dibuthyl ether, and separation of furfural from the ether phase by distillation.

2. In the process of claim 1 where the ionic liquids component N-alkyl imidazole contains as the alkyl group methyl, ethyl, propyl or buthyl.

3. In the process of claim 1 where the quantity of N alkylimidazolium sulphate, N-alkyl imidazolium chloride, water, cellulose, sulphuric acid, dibuthyl ether can vary in the mole proportions, namely the proportions of N-alkyl imidazolium sulphate to N-alkyl imidazolium chloride can vary from 1 to 0.01 up to 1 to 0.5, the quantity of water may vary from 1 to 20% of the ionic reaction phase, the quantity of additional sulphuric acid may vary between 1 to 30% of the ionic reaction phase, the quantity of dibuthyl ether is not critical for the reaction but should stay as 10 to 100% of the ionic reaction phase.

4. In the process of claim 1 where the amount of cellulose in the ionic reaction phase is 1% to 15%.

5. In the process of claim 1 where the reaction time is 20 minutes to 180 minutes, the temperature 80 to 250° C., where the higher temperature reduces the reaction time but increases the side products, so that preferably the time should be 30 minutes to one hour for a temperature of 80° C. to 160° C.

6. In the process of claim 1 where furfural and dibuthyl ether are separated by a known distillation technique with a number of theoretical plates and reflux according to the purity desired, considering that the dibuthyl ether as an impurity for the application of furfural as a fuel component is irrelevant.

7. In the process of claim 1 where the dialkyl ether used for the continuous extraction should have a boiling point as low as possible as compared to furfural in order to reduce the temperatures in the distillation columns as well as the number of plates and should have a boiling point as near as possible to the reaction temperature in order to avoid to work at higher than atmospheric pressure during the extraction, chosen from dialkyl ethers where one alkyl group is methyl, ethyl, propyl, butyl, pentyl, hexyl and the second group should have the corresponding number of carbon atoms in order that the boiling point of the ether at atmospheric pressure is similar to the reaction temperature.
